(19) United States
(12) Patent Application Publication
(10) Pub. No.: US 2003/0167054 A1
(43) Pub. Date: Sep. 4, 2003

(54) Device for cell implantation

(76) Inventors: Lawrence Rosenberg, Montreal (CA); Toshia Fujisato, Osaka (JP)

Correspondence Address: OGILVY RENAULT, 1981 MCGILL COLLEGE AVENUE, SUITE 1600, MONTREAL, QC, H3A2Y3, CA

(21) Appl. No.: 10/275,074

(22) PCT Filed: May 4, 2001
(86) PCT No.: PCT/CA01/00630

(51) Int. Cl. A61F 2/04

(52) U.S. Cl. 604/891.1; 424/424; 514/866

(57) ABSTRACT

A device for cell implantation has a housing containing a matrix which isolates the cells in the housing; the housing has a window which comprises a membrane which permits passage of nutrients for the cells into the matrix from a location externally of the housing; the housing has an injection port permitting injection of cells into the matrix in the housing. The device may be employed, for example, to facilitate islet implantation in the treatment of diabetes mellitus.

### TECHNICAL FIELD

The invention relates to the development of a device to facilitate cell implantation, for example, islet implantation in treatment for diabetes mellitus.

### BACKGROUND ART

Diabetes Mellitus

Diabetes mellitus has been classified as type I, or insulin-dependent diabetes mellitus (IDDM) and type II, or non-insulin-dependent diabetes mellitus (NIDDM). NIDDM patients have been subdivided further into (a) nonobese (possibly IDDM in evolution), (b) obese, and (c) maturity onset (in young patients). Among the population with diabetes mellitus, about 20% suffer from IDDM. Diabetes develops either when a diminished insulin output occurs or when a diminished sensitivity to insulin cannot be compensated for by an augmented capacity for insulin secretion. In patients with IDDM, a decrease in insulin secretion is the principal factor in the pathogenesis, whereas in patients with NIDDM, a decrease in insulin sensitivity is the primary factor. The mainstay of diabetes treatment, especially for type I disease, has been the administration of exogenous insulin.

Rationale For More Physiologic Therapies

Tight glucose control appears to be the key to the prevention of the secondary complications of diabetes. The results of the Diabetes Complications and Control Trial (DCCT), a multicenter randomized trial of 1441 patients with insulin dependent diabetes, indicated that the onset and progression of diabetic retinopathy, nephropathy, and neuropathy could be slowed by intensive insulin therapy (The Diabetes Control and Complication Trial Research Group. The effect of intensive treatment of diabetes on the development and prgoression of long-term complications in insulin-dependent diabetes mellitus. N Engl J Med 1993, 29:977-986). Strict glucose control, however, was associated with a three-fold increase in incidence of severe hypoglycemia, including episodes of seizure and coma. As well, although glycosylated hemoglobin levels decreased in the treatment group, only 5% maintained an average level below 6.05% despite the enormous amount of effort and resources allocated to the support of patients on the intensive regime (The Diabetes Control and Complication Trial Research Group. The effect of intensive treatment of diabetes on the development and prgoression of long-term complications in insulin-dependent diabetes mellitus. N Engl J Med 1993, 29:977-986). The results of the DCCT clearly indicated that intensive control of glucose can significantly reduce (but not completely protect against) the long-term microvascular complications of diabetes mellitus.

The delivery of insulin in a physiologic manner has been an elusive goal since insulin was first purified by Banting , Best, McLeod and Collip. Even in a patient with tight glucose control, however, exogenous insulin has not been able to achieve the glucose metabolism of an endogenous insulin source that responds to moment-to-moment changes in glucose concentration and therefore protects against the development of microvascular complications over the long term.

A major goal of diabetes research, therefore, has been the development of new forms of treatment that endeavor to reproduce more closely the normal physiologic state. One such approach, a closed-loop insulin pump coupled to a glucose sensor, mimicking β-cell function in which the secretion of insulin is closely regulated, has not yet been successful. Only total endocrine replacement therapy in the form of a transplant has proven effective in the treatment of diabetes mellitus. Although transplants of insulin-producing tissue are a logical advance over subcutaneous insulin injections, it is still far from clear whether the risks of the intervention and of the associated long-term immunosuppressive treatment are lower than those in diabetic patients under conventional treatment.

Despite the early evidence of the potential benefits of vascularized pancreas transplantation, it remains a complex surgical intervention, requiring the long-term administration of chronic immunosuppression with its attendant side effects. Moreover, almost 50% of successfully transplanted patients exhibit impaired tolerance curves (Wright F H, Smith J L, Ames S A, Scanbacher B, Corry R J. Arch Surg 1989; 124:796-799; Landgraft R, Nusser J, Riepl R L, Fiedler F, Illner W D, Abendroth D, Land W. Diabetologia 1991; 34 (suppl 1):S61; Morel P, Brayman K L, Goetz F C, Kendall D M, Moudry-Munns K, Chau C. Transplantation 1991;51:990-1000), raising questions about their protection against the long-term complications of chronic hyperglycemia.

The major complications of whole pancreas transplantation, as well as the requirement for long term immunosuppression, has limited its wider application and provided impetus for the development of islet transplantation. Theoretically, the transplantation of islets alone, while enabling tight glycemic control, has several potential advantages over whole pancreas transplantation. These include the following: (i) minimal surgical morbidity, with the infusion of islets directly into the liver via the portal vein; (ii) the possibility of simple re-transplantation for graft failures; (iii) the exclusion of complications associated with the exocrine pancreas; (iv) the possibility of modifying islets in vitro prior to transplantation to reduce their immunogenicity; and (vi) the ability to encapsulate islets in artificial membranes to isolate them from the host immune system. Moreover, isolated islets lend themselves to cryopreservation and banking in association with a careful and standardized quality control program before the implantation.

Current Problems With Islet Transplantation

Adequate numbers of isogeneic islets transplanted into a reliable implantation site can only reverse the metabolic abnormalities in diabetic recipients in the short term. In those that were normoglycemic post-transplant, hyperglycemia generally recurred within 3-12 months. multifactorial (Rosenberg L. Clinical islet transplantation. Int'l J Pancreatology 24:145-168, 1998). The return of the diabetic state that occurs with time is multifactorial (Rosenberg L Int'l J Pancreatology 24:145-168, 1998), and include inadequate vascularization, lack of trophic support and cell destruction due to allo- and/or autoimmune destruction. In large measure due to the emphasis on the immunological issues, considerable enthusiasm has been generated for the immunoisolation of islets, an approach which would potentially also eliminate the need for systemic immunosuppression.

The reasons are compelling. Immunosuppression is harmful to the recipient, and may impair islet function and possibly cell survival. Unfortunately, micro-encapsulated islets injected into the peritoneal cavity of the dog fail within 6 months, and islets situated in a biohybrid device placed in series with the circulation, also fail, but at about one year. In each instance, however, histological evaluation of the graft has indicated a substantial loss of islet cell mass. No reasons have been advanced for these changes.

All these constructs and the variations thereof, have been designed to produce an immuno-barrier so that cells could be transplanted without the requirement for immunosuppression. However, these have not proven 100% reliable in this regard, and have certainly not contributed to the long-term maintenance of islet cell function and survival Therefore maintenance of an effective islet cell mass post-transplantation remains a significant problem.

Requirements For Islet Cell Survival And the Consequences of Islet Isolation For Transplantation

Generally, the maintenance of cell survival in-situ is dependent on factors in the local microenvironment—growth factors, cell-cell interrelationships and cell-matrix interactions. In a series of studies (Wang R N, Paraskevas S, Rosenberg L. J Histochem Cytochem 1999;47:499-506; Ilieva A, Yuan S, Wang R N, Agapitos D, Hill D J, Rosenberg L. J Endocrinol 1999;161:357-364; Wang R N, Rosenberg L. J Endocrinol 1999;163:181-190), it has been demonstrated that the process of islet isolation leads to the abrogation of each of these support mechanisms. As a consequence, shortly after isolation, islet cells begin to undergo programmed cell death (Rosenberg L, Wang R N, Paraskevas S, Maysinger D. Surgery 1999;126:393-398). It is therefore not surprising that the transplantation of purified islets might ultimately cease to function.

### DISCLOSURE OF THE INVENTION

The aim of the invention is to provide a platform for the delivery of cells, for example, islet cells wherein a device is pre-vascularized prior to cell implantation. Furthermore, the design of the device is such that it will: (a) further support cell survival by allowing for co-localization of required growth factors and/or other cell types; and (b) allow easy access for re-seeding of the device with additional cells should the need arise following implantation.

There is thus provided a biocompatible cell delivery device and a method for the construction of a such a device.

In accordance with one aspect of the invention there is provided a device for cell implantation in a recipient comprising: a housing containing a matrix adapted to isolate in said housing cells having a desired biological function, at least one window in said housing, said window comprising a membrane permitting passage into said matrix of nutrients for said cells, and at least one port in said housing for injection of cells into said matrix.

In accordance with another aspect of the invention there is provided a device of the invention for use in treating diabetes mellitus.

In another aspect of the invention there is provided a method of treating diabetes mellitus comprising implanting in a recipient having need for treatment of diabetes mellitus, a device of the invention, allowing said matrix to vascularize and injecting islets into the vascularized matrix through the at least one port.

### DESCRIPTION OF BEST MODES

If cells, for example islet cells, are to survive following transplantation then a microenvironment that provides the required trophic support needs to be supplied. In addition, cells must be placed into a site that is well vascularized in order to benefit immediately from the availability of oxygen and other necessary nutrients.

It has already been demonstrated that isolated islets survive well in a solid three-dimensional matrix such as a collagen gel (Rosenberg L, Metrakos P, Rajotte R. Transpl Proc 1992; 24:3012-3013), and that co-localization with cells, such as duct epithelial cells, that secrete growth factors, such as insulin-like growth factor-2 (IGF-2), further enhances cell survival (Ilieva A, Yuan S, Wang R N, Agapitos D, Hill D J, Rosenberg L. J Endocrinol 1999;161:357-364). It has also been demonstrated previously that when basic fibroblast growth factor (bFGF)is added to a collagen scaffold on which chondrocytes were seeded, the regeneration of cartilege was accelerated (Fujisato T, Sajiki T, Liu Q, Ikada Y. Biomaterials 1996;17:155-62). This has been attributed to the rapid formation of a capillary network involving the scaffold at an early stage of transplantation.

The present invention provides a biocompatible cell delivery device that can be pre-vascularized prior to cell seeding. The device provides a customizable micro-environment with respect to supporting cells, matrix and growth factors to support specific cell types. In addition, because the seeded cells are localized to a defined space, they are not lost during implantation; all cells end up where they need to be, and hence fewer cells are required to achieve a functional outcome. Design parameters allow for: (a) customization of shape and size; (b) the re-seeding of the device should that become necessary after implantation; and (c) for easy removal of the device should this be required.

### DESCRIPTION OF PREFERRED EMBODIMENTS

Suitably the at least one window comprises a polyvinyl alcohol membrane supported by a biocompatible fabric. The membrane permits passage of nutrients for the cells, into the matrix.

In one embodiment the housing comprises a substantially annular wall surrounding the matrix and a pair of generally circular windows extends between spaced apart opposed edges of the wall, so that the windows are in opposed, spaced apart, facing relationship.

Suitably the matrix contains biological entities which will vascularize the device, for example, fibroblasts and bFGF. A polyglycoride cloth is a suitable matrix when impregnated with a collagen gel.

It will be understood that the cells may be any hormone secreting cells needed in therapy or treatment of a disease or disorder. In the treatment of diabetes mellitus the cells comprise islets.

### BRIEF DESCRIPTION OF DRAWINGS

The invention is illustrated by reference to the accompanying drawings in which:

FIG. 1 is an exploded view of the device;

FIG. 2 shows the device implanted subcutaneously in a nude mouse;

FIGS. 3A and 3B shows a section through the vascularized interiors of implanted devices, at 2 weeks.

FIG. 4 shows low power (upper panel) and high power (lower panel) views representing a histological section taken through the center of a device that has been pre-vascularized following implantation into a normoglycemic hamster, and then seeded with hamster islets (stained brown with aldehyde fuschin-indicating the presence of insulin). This demonstrates that β-cell function and survival is well maintained after two months employing the device of FIG. 1 seeded with syngeneic islets;

FIG. 5 demonstrates that the device of FIG. 1 seeded with canine islets and transplanted in diabetic nude mice, functions efficiently to reverse the diabetic state.

### DESCRIPTION OF PREFERRED EMBODIMENTS WITH REFERENCE TO DRAWINGS

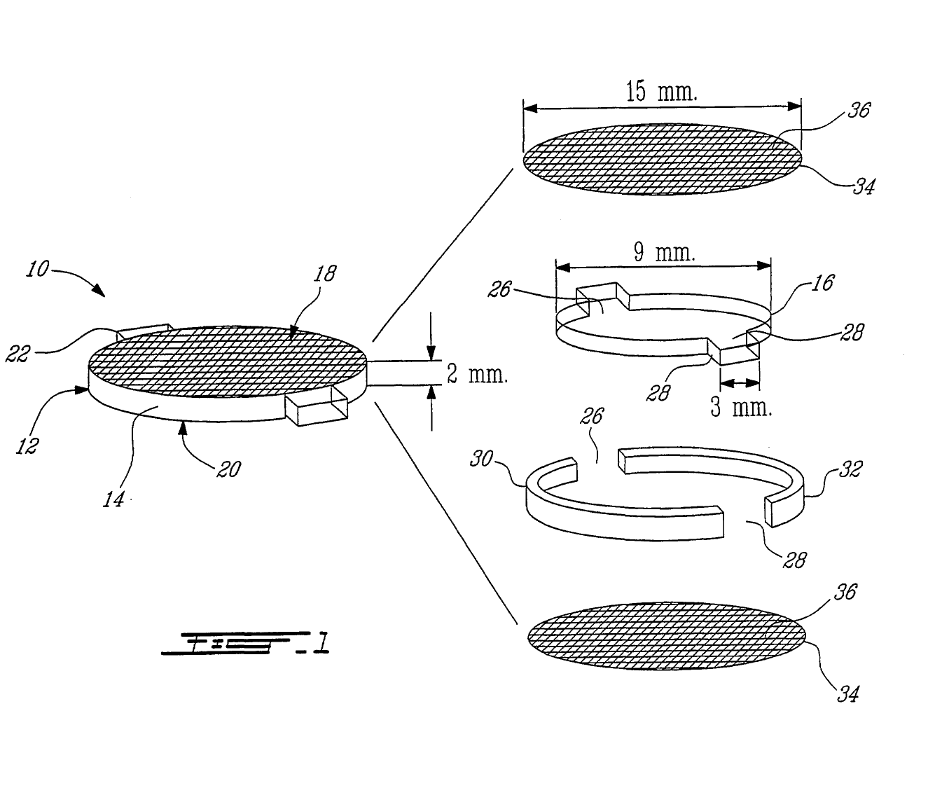

With further reference to FIG. 1, an implant device 10 comprises a housing 12 having an annular wall 14, a matrix 16 and windows 18 and 20.

Matrix projections 22 and 24 extend from ports 26 and 28 respectively in wall 14.

As shown in FIG. 1, wall 14 is formed from segments 30 and 32 of a silicone ring, and the matrix 16 is of cloth, for example, a polyglycoride cloth.

The windows 18 and 20 each comprise a support mesh 34 typically of polyester covered with a PVA (polyvinyl alcohol) membrane 36.

### EXAMPLE

A scaffold or matrix 16 is prepared as illustrated in FIG. 1. The matrix 16 is created from a 1 mm polyglycoride (PGA) cloth (Gunze Co. Ltd., Kyoto, Japan) cut to desired shape and size (the example provides for a round shape of 9 mm diameter) with at least two projections 22 and 24 for guided tissue in-growth.

The scaffold or matrix 16 is placed inside an outer shell or housing 12 comprising a pair of polyvinyl alcohol (PVA) membranes and an implant grade silicone ring which forms wall 14 (Applied Silicone Corp., Ventura, Calif.) having at least two openings or ports 26 and 28 for projections 22 and 24, respectively. The PVA membranes 36 were prepared from a 3% aqueous solution of molecular weight 70,000-100,000 (Sigma Chemical Co., St. Louis, Mo.), by a cross-linking reaction in glutaraldehyde acidic solution, and were reinforced by polyester mesh 34 of 105 μm opening (Spectrum Lab Inc., Laguna Hill, Calif.) pretreated with a corona discharge to be hydrophilic using a high voltage high frequency single ended plasma driver (Scientific Systems, Amherst, N.H.). The construct is then scaled with implant grade silicone.

In the example provided, 1 μg/ml bFGF (Becton Dickinson Labware, Bedford, Mass.) and/or skin-derived fibroblasts (in the example, these are of hamster origin) in collagen solution are injected through a port 26 or 28 into matrix 16, whereupon gelation occurs inside the device 10.

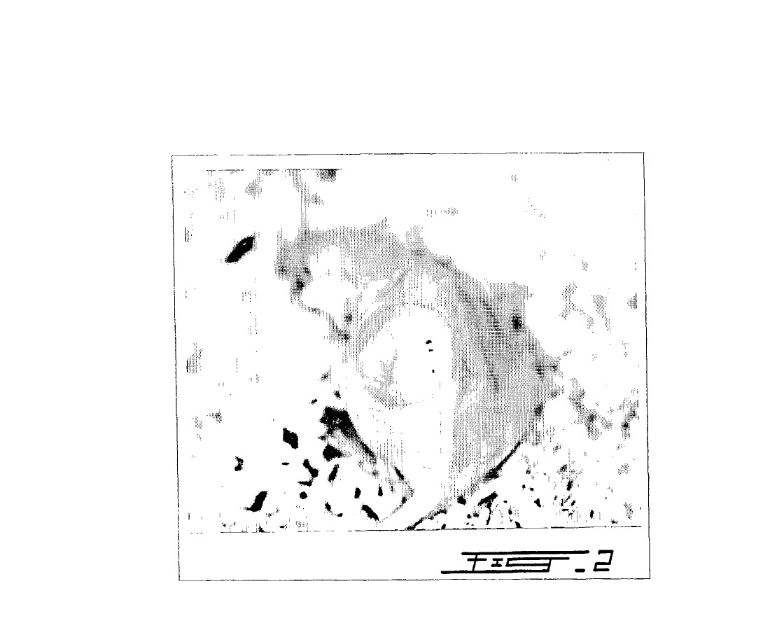

The device can be implanted into a suitable location, for example, subcutaneously or intra-peritoneally. In a particular example (FIG. 2), the device 10 was implanted subcutaneously. Vascular ingrowth into the device at 1 week following implantation was observed. Differences seem less apparent after 2 weeks.

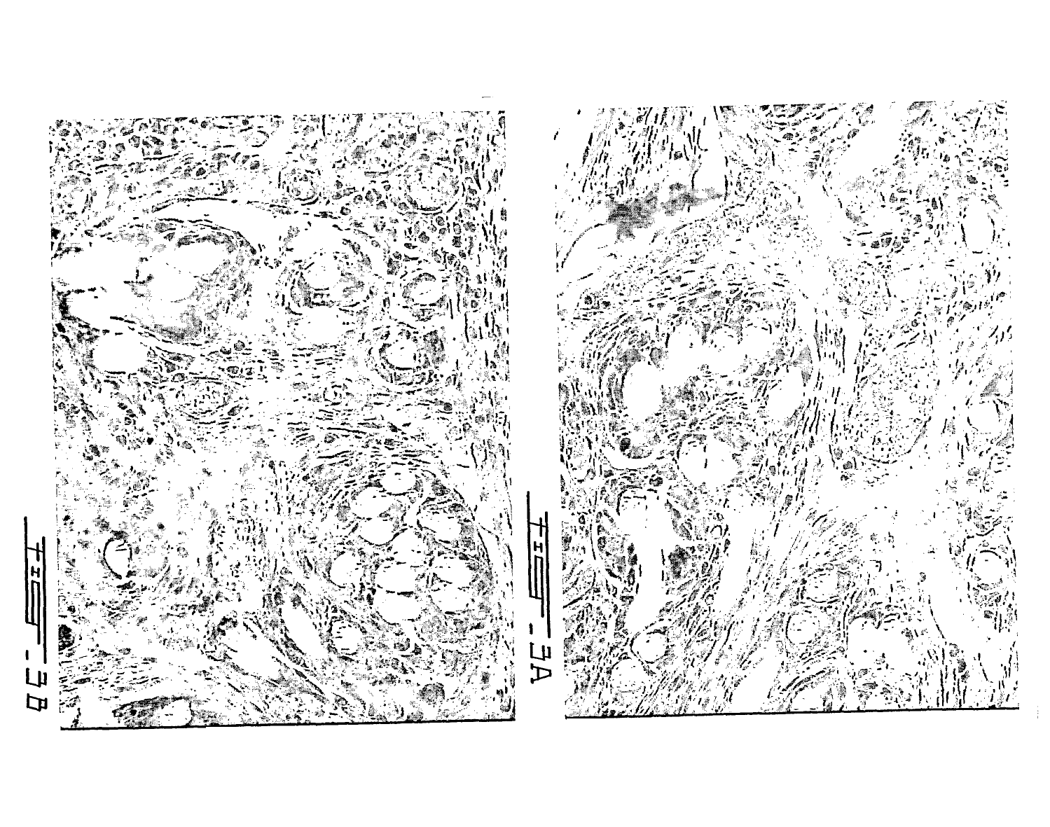

More especially FIG. 3A shows the vascularized tissue inside the matrix of collagen coated polyglycoride cloth two weeks after subcutaneous implant of the device of FIG. 1 in a hamster, where the matrix contains bFGF alone. FIG. 3B shows the corresponding vascularized tissue when the matrix contains both bFGF and fibroblasts.

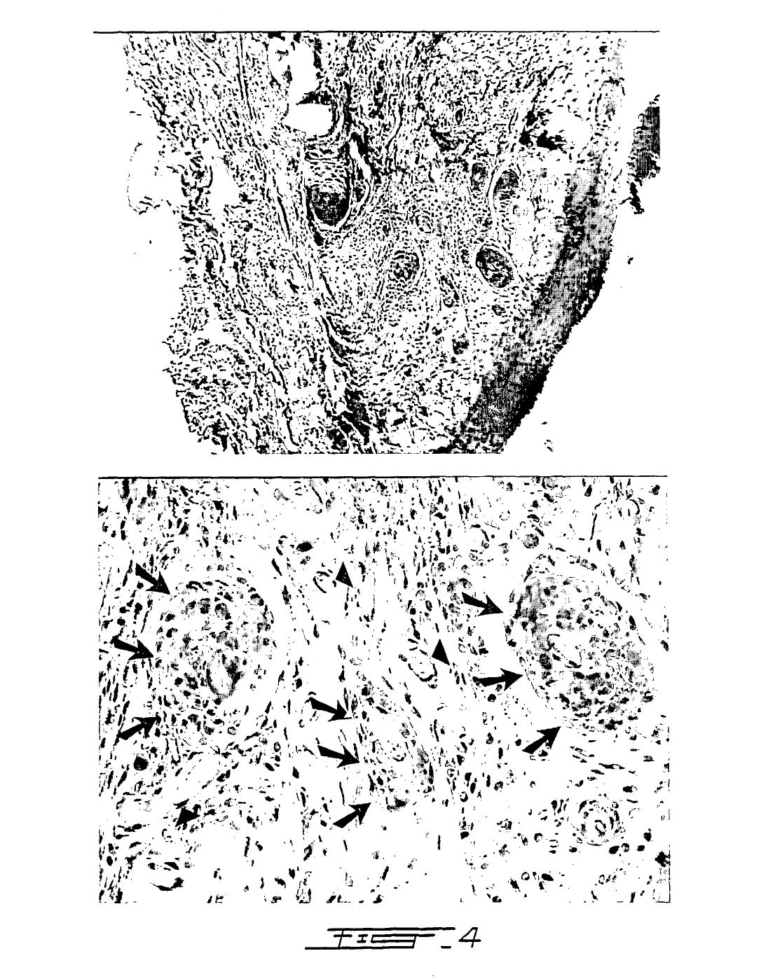

Implantation into normoglyceimic Syrian golden hamsters, of devices seeded with syngeneic islets, demonstrates that islet cell function and survival is well maintained for a period at least 2 months (FIG. 4). In FIG. 4, the arrowheads shows blood vessels and the arrows indicate islets.

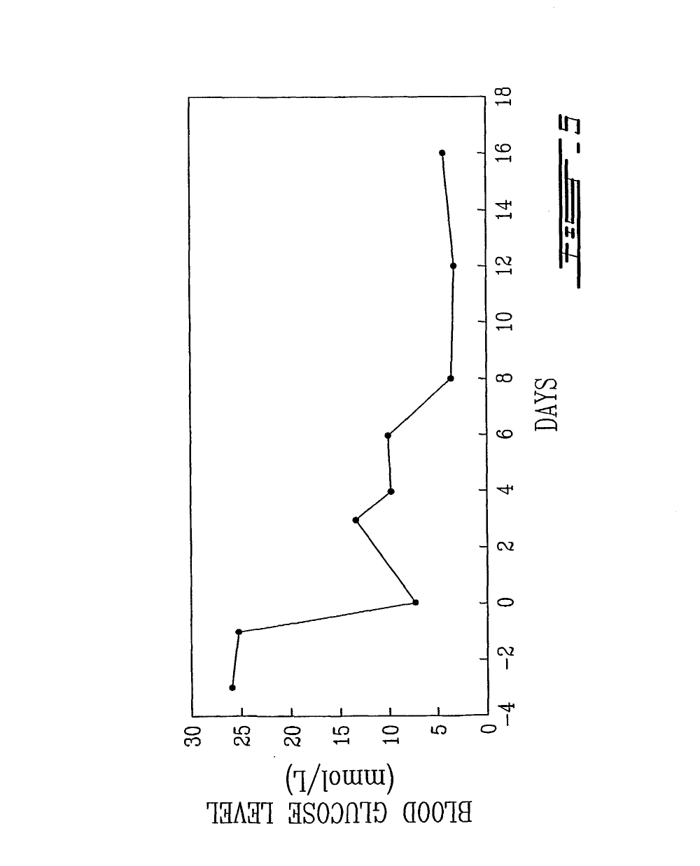

A further example (FIG. 5) demonstrates that devices seeded with canine islets and transplanted into diabetic nude mice, function efficiently to reverse the diabetic state; more especially FIG. 5 shows the change of blood glucose level in the STZ-diabetic nude mouse following transplantation of canine islets into a pre-vascularized scaffold device of the invention.

1. A device for cell implantation in a recipient comprising:
a housing containing a matrix adapted to isolate in said housing cells having a desired biological function, at least one window in said housing, said window comprising a membrane permitting passage into said matrix of nutrients for said cells, and
at least one port in said housing for injection of cells into said matrix.

2. A device according to claim 1, wherein said at least one window comprises a polyvinyl alcohol membrane.

3. A device according to claim 1 or 2, wherein said at least one window further comprises a biocompatible fabric which supports said membrane.

4. A device according to claim 3, wherein said biocompatible fabric is of polyester mesh.

5. A device according to claim 1, 2, 3 or 4, wherein said housing comprises a substantially annular wall surrounding said matrix and said at least one window comprises a pair of generally circular opposed windows extending between the spaced apart opposed edges of said wall.

6. A device according to claim 1, 2, 3, 4 or 5, wherein said matrix contains biological entities adapted to vascularize said device.

7. A device according to claim 6, wherein said entities comprise one or more of fibroblasts and bFGF.

8. A device according to claim 1, 2, 3, 4, 5, 6 or 7, wherein said matrix comprises a polyglycoride cloth supporting a collagen gel.

9. A device according to claim 1, 2, 3, 4, 5, 6, 7 or 8, wherein said matrix is vascularized and further including cells.

10. A device according to claim 9, wherein said cells are islets for treatment of diabetes mellitus.

11. A device of any one of claims 1 to 10, for use in the treatment of diabetes mellitus.

12. A method of treating diabetes mellitus comprising implanting in a recipient having need for treatment of diabetes mellitus, a device as defined in any one of claims 1 to 10, allowing said matrix to vascularize and injecting islets into the vascularized matrix through the at least one port.
